(19) United States
(12) Patent Application Publication
(10) Pub. No.: US 2019/0185404 A1
(43) Pub. Date: Jun. 20, 2019

(54) PROCESSES FOR THE HYDROGENATION OF PHTHALATE ESTERS

(71) Applicants: Nicolaas A. De Munck, Barendrecht (NL); Hans K. T. Goris, Zaventem (BE); Eddy Van Driessche, Eeklo (BE)

(72) Inventors: Nicolaas A. De Munck, Barendrecht (NL); Hans K. T. Goris, Zaventem (BE); Eddy Van Driessche, Eeklo (BE)

(21) Appl. No.: 16/323,479

(22) PCT Filed: Aug. 24, 2017
(86) PCT No.: PCT/EP2017/071283

§ 371 Date: Feb. 5, 2019

(30) Foreign Application Priority Data
Sep. 9, 2016 (EP) 16188173.5

(51) Int. Cl. C07C 51/36; C07C 67/303

(52) U.S. Cl. CPC C07C 51/36; C07C 67/303

(57) ABSTRACT

A process for ring hydrogenation of a benzenepolycarboxylic acid or derivative thereof, which process comprises contacting a feed stream comprising said acid or derivative thereof with a hydrogen-containing gas in the presence of a catalyst under hydrogenation conditions to produce a hydrogenated product, wherein said catalyst comprises a Group VIII metal, a support material and a halogen, and wherein the halogen is present in an amount of from 0.02 to 0.60% by weight, based on the total weight of the catalyst.

### FIELD OF THE INVENTION

The present invention relates to a process for the hydrogenation of benzenepolycarboxylic acids and derivatives thereof, and to supported catalysts for the hydrogenation of benzenepolycarboxylic acids and derivatives thereof. More particularly, the present application relates to a process for the ring-hydrogenation of benzenepolycarboxylic acids and derivatives thereof utilizing a supported transition metal catalyst.

### BACKGROUND OF THE INVENTION

Hydrogenation is an established process both in the chemical and petroleum refining industries. Hydrogenation is conventionally carried out in the presence of a catalyst, which usually comprises a metal hydrogenation component deposited on a porous support material. The metal hydrogenation component is often one or more metals, for example nickel, platinum, palladium, rhodium, ruthenium or mixtures thereof.

Many organic compounds have one or more groups or functionality that is susceptible to hydrogenation under appropriate conditions with the use of a suitable metal containing catalyst. One particular group of compounds that are susceptible to hydrogenation is those that contain one or more unsaturated groups or functionality such as for example carbon-carbon double bonds or triple bonds.

Hydrogenated derivatives of benzenepolycarboxylic acids or derivatives thereof, such as esters and/or anhydrides, have many uses. Of particular interest is their use as plasticisers for polymeric materials. In this context, the dialkyl hexahydrophthalates are of particular interest. These materials may be produced by hydrogenation of the corresponding phthalic acid ester in the presence of a hydrogen-containing gas and an active metal hydrogenation catalyst deposited on a support. Processes for the hydrogenation of benzenepolycarboxylic acids and their derivatives in the presence of a catalyst comprising ruthenium supported on an aluminum oxide or silicon dioxide supports are disclosed for example in U.S. Pat. Nos. 6,284,917 and 7,208,545 that exemplify the use of catalysts prepared by impregnating an aluminum oxide support with an aqueous ruthenium (III) nitrate solution.

U.S. Pat. No. 7,355,084 discloses a process for hydrogenating an aromatic organic compound with hydroxyl or amino group bound to the aromatic ring by contact with a hydrogen-containing gas in the presence of a halogen-free catalyst comprising ruthenium supported on silicon dioxide to form the corresponding cycloaliphatic compound. The catalyst is prepared by treating the support material with a halogen-free aqueous solution of a low molecular weight ruthenium compound, for example ruthenium (III) nitrosyl nitrate, ruthenium (III) acetate or an alkali metal ruthenate (IV). The ruthenium precursors are exclusively ruthenium compounds with no chemically bound halogen. It is disclosed that the halogen-free solution should contain no halogen, or less than 500 ppm halogen, so that the catalyst has a chlorine content of below 0.05% by weight, based on the total weight of the catalyst.

U.S. Pat. No. 7,618,917 discloses the use of a similar catalyst in xylose hydrogenation. According to that document the observed high activity and selectivity of the catalysts can be attributed to the virtual absence of halogen in the catalyst.

A catalyst for the hydrogenation of carbocyclic aromatic groups to carbocyclic aliphatic groups is disclosed in US patent application no. US2010/0152436. The catalyst is a shell catalyst prepared by impregnation of silicone dioxide support with a solution of ruthenium acetate. The support material and the impregnation solution are halogen-free, especially chlorine-free, meaning that the content of halogen in the support material and in the impregnation solution comprises is less than 500 ppm halogen by weight. The halogen content of the catalyst is preferably from 0 to less than 80 ppm based on the total weight of the catalyst.

U.S. Pat. No. 7,595,420 discloses a process for hydrogenating benzenepolycarboxylic acids comprising contacting the compound with hydrogen-containing gas in the presence of a catalyst comprising one or more catalytically active metals including ruthenium and nickel applied to a silicate or aluminosilicate mesoporous zeolite such as MCM-41, MCM-48 and MCM-50. The catalysts are prepared by deposition/formation of one or more organic metal complexes on or in the support followed by decomposition of said complexes. According to the examples the organic metal complexes are obtained by combining ruthenium nitrosyl nitrate with triethanol amine.

U.S. Pat. No. 6,803,341 discloses a method for preparing dimethyl 1, 4-cyclohexanedicarboxylate by catalytic hydrogenation of dimethyl terephthalate using an alumina-supported ruthenium catalyst. The catalyst is prepared by impregnating alumina with a solution of ruthenium (III) chloride followed by calcining and reduction at a high temperature of 450-500° C. The chloride content of the catalyst is not disclosed, but is expected to be as high as 2.06 wt % based on the total catalyst weight before calcination.

Severe calcining and reduction conditions may lead to removal of the all chloride by hydrochloric acid gas formation.

There remains a need for new, efficient hydrogenation processes for the hydrogenation of benzenepolycarboxylic acids and derivatives thereof, and in particular for the ring-hydrogenation of benzenepolycarboxylic acids and derivatives thereof, which processes are highly selective and proceed at good reaction rates. Additionally, there remains a need for new catalysts for use in such processes, and in particular, efficient catalysts that can be prepared simply and cheaply from readily available starting materials. It is therefore an object of the invention to provide a process for hydrogenating benzenepolycarboxylic acids and derivatives thereof to hydrogenation products with high levels of conversion, selectivity and with good rates of reaction, and to provide a hydrogenation catalyst for use in such a hydrogenation process.

### SUMMARY OF THE INVENTION

It has been found that the catalytic hydrogenation of benzenepolycarboxylic acids and derivatives thereof is less sensitive to the presence of halogens in the catalyst than previously thought. Surprisingly, contacting a benzenepolycarboxylic acids and derivatives thereof with a hydrogen containing gas in the presence of a supported transition metal catalyst comprising a halogen provides an efficient and highly active process for the ring-hydrogenation of the benzenepolycarboxylic acids and derivatives thereof.

The present invention therefore provides a process for ring hydrogenation of a benzenepolycarboxylic acid or derivative thereof, which process comprises contacting a feed stream comprising said acid or derivative thereof with a hydrogen containing gas in the presence of a catalyst under hydrogenation conditions to produce a hydrogenated product, wherein said catalyst comprises a Group VIII metal (previous IUPAC version of the periodic table of the elements), a support material and a halogen, wherein the halogen is present in an amount of at least 0.02% by weight, based on the total weight of the catalyst, and preferably from 0.02 to 0.60% by weight.

The Group VIII metal is preferably rhodium, ruthenium, platinum, palladium or mixtures thereof. A particularly preferred metal is ruthenium.

The support materials are preferably chosen from alumina, silica or mixtures thereof with the most preferred material being silica.

### DETAILED DESCRIPTION OF THE INVENTION

In the process of the present invention benzenepolycarboxylic acids or derivatives thereof are hydrogenated to the corresponding cyclohexyl derivative in the presence of a hydrogen-containing gas under hydrogenation conditions, wherein said catalyst comprises a Group VIII metal, a support material and a halogen, and wherein the halogen is present in an amount of from 0.02 to 0.60% by weight, based on the total weight of the catalyst. We have found that a catalyst comprising a Group VIII metal, a support material and a halogen provides a highly active and efficient catalyst for the hydrogenation of benzenepolycarboxylic acids and derivatives thereof when the halogen, for example chlorine, is present in an amount at least 0.03 wt %, preferably of from 0.06 to 0.50 wt %, based on the total weight of the catalyst, more preferably between 0.10 and 0.50. Contents of halogen in a range of from 0.20 and 0.40% by weight, based on the total weight of the catalyst provide best compromises between the economics of the preparation process and the catalyst performances.

The term “benzenepolycarboxylic acid or a derivative thereof” used for the purposes of the present invention encompasses all benzenepolycarboxylic acids as such, e.g. phthalic acid, isophthalic acid, terephthalic acid, trimellitic acid, trimesic acid, hemimellitic acid and pyromellitic acid, and derivatives thereof, particularly monoesters, diesters and possibly triesters and tetraesters, in particular alkyl esters, and anhydrides such as phthalic anhydride or trimellitic anhydride or their esters. The esters used are alkyl, cycloalkyl and alkoxyalkyl esters, where the alkyl, cycloalkyl and alkoxyalkyl groups generally have from 1 to 30, preferably from 2 to 20 and particularly preferably from 3 to 18, carbon atoms and can be branched or linear. Preferably, the benzenepolycarboyxlic acid or derivative thereof is a C7-C13 dialkyl phthalate, or a C7-C13 dialkyl terephthalate, or a mixture thereof. Also suitable are alkyl terephthalates, alkyl phthalates, alkyl isophthalates in which one or more of the alkyl groups contain 5, 6 or 7 carbon atoms (e.g. are C5, C6 or C7 alkyl groups).

Such compounds are well known by the skilled person and examples thereof can be found in U.S. Pat. No. 7,732,634, the disclosure of which is incorporated herein by reference.

Also suitable are esters in which the alkyl groups of the ester are different alkyl groups. Mixtures of one or more of alkyl esters may be used.

Also suitable are anhydrides of phthalic acid, trimellitic acid, hemimellitic acid and pyromellitic acid.

Envisaged as examples of compounds in which the alkyl groups are not identical are butylpropyl terephthalate or compounds where one of the alkyl groups is replaced by a benzyl group such as for example in butylbenzyl terephthalate.

In the process of the present invention it is also possible to use mixtures of one or more of the benzenepolycarboxylic acid or derivatives thereof described herein. When the derivatives are esters the mixture may be derived through use of two or more alcohols in admixture or in sequence to esterify the same sample of a benzenepolycarboxylic acid derivative or a mixture of two or more benzenepolycarboxylic acids. Alternatively the alcohols may be used to form, in separate syntheses, two different esterified derivatives, which may then be mixed together to form a mixture of two or more esterified derivatives. In either approach the mixture may comprise a mixture of esters derived from branched or linear alcohols, for example the mixture may comprise ester derivatives prepared from C₇, C₉, C₈, C₁₀ and C₁₁ linear or branched alcohols, preferably linear alcohols, with the alcohols being used in the same synthesis of a mixture of derivatives or in separate syntheses of the derivative where the resultant derivative products in each synthesis are combined to form a mixed derivative. Preferably, the benzenepolycarboyxlic acid or derivative thereof comprises a mixture of C₇ dialkyl phthalates and C₉ dialkyl phthalates, a mixture of C₇ dialkyl terephthalates and C₉ dialkyl terephthalates, a mixture of C₇ dialkyl phthalates and C₁₀ dialkyl phthalates, or a mixture of C₇ dialkyl terephthalates and C₁₀ dialkyl terephthalates.

In the process of the present invention the preferred hydrogenation products are those derived from phthalates and in particular the following: cyclohexane-1,2-dicarboxylic acid di(isopentyl) ester, obtainable by hydrogenation of a di(isopentyl) phthalate having the Chemical Abstracts registry number (in the following: CAS No.) 84777-06-0; cyclohexane-1,2-dicarboxylic acid di(isoheptyl) ester, obtainable by hydrogenating the di(isoheptyl) phthalate having the CAS No. 71888-89-6; cyclohexane-1,2-dicarboxylic acid di(isononyl) ester, obtainable by hydrogenating the di(isononyl)phthalate having the CAS No. 68515-48-0; cyclohexane-1,2-dicarboxylic acid di(isononyl) ester, obtainable by hydrogenating the di(isononyl)phthalate having the CAS No. 28553-12-0, which is based on n-butene; cyclohexane-1,2-dicarboxylic acid di(isononyl) ester, obtainable by hydrogenating the di(isononyl)phthalate having the CAS No. 28553-12-0, which is based on isobutene; a 1,2-di-C9-ester of cyclohexanedicarboxylic acid, obtainable by hydrogenating the di(nonyl)phthalate having the CAS No. 68515-46-8; cyclohexane-1,2-dicarboxylic acid di(isodecyl) ester, obtainable by hydrogenating a di(isodecyl)phthalate having the CAS No. 68515-49-1; 1,2-C7-11-ester of cyclohexanedicarboxylic acid, obtainable by hydrogenating the corresponding phthalic acid ester having the CAS No. 68515-42-4; 1,2-di-C7-11-ester of cyclohexanedicarboxylic acid, obtainable by hydrogenating the di-C7-11-phthalates having the following CAS Nos.: 111381-89-6, 111381-90-9, 111381-91-0, 68515-44-6, 68515-45-7 and 3648-20-7; a 1,2-di-C9-11-ester of cyclohexanedicarboxylic acid, obtainable by hydrogenating a di-C9-11-phthalate having the CAS No. 98515-43-5; a 1,2-di(isodecyl)cyclohexanedicarboxylic acid ester, obtainable by hydrogenating a di(isodecyl)phthalate, consisting essentially of di-(2-propylheptyl)phthalate; 1,2-di-C7-9-cyclohexanedicarboxylic acid ester, obtainable by hydrogenating the corresponding phthalic acid ester, which comprises branched and linear C7-9-alkylester groups; respective phthalic acid esters which may be e.g. used as starting materials have the following CAS Nos.: di-C7-9-alkylphthalate having the CAS No. 111 381-89-6; di-C7-alkylphthalate having the CAS No. 68515-44-6; and di-C9-alkylphthalate having the CAS No. 68515-45-7.

More preferably, the above explicitly mentioned C5-7, C9, C10, C7-11, C9-11 and C7-9 esters of 1,2-cyclohexanedicarboxylic acids are the hydrogenation products of the commercially available benzenepolycarboxylic acid esters with the trade names Jayflex® DINP (CAS No. 68515-48-0), Jayflex DIDP (CAS No. 68515-49-1), Jayflex DIUP (CAS No. 85507-79-5), Jayflex DTDP (CAS No. 68515-47-9), Jayflex L911P (CAS No. 68515-43-5), Vestinol® 9 (CAS No. 28553-12-0), TOTM-I® (CAS No. 3319-31-1), Linplast® 68-TM and Palatinol N (CAS No. 28553-12-0) which are used as plasticizers in plastics.

Further examples of commercially available benzenepolycarboxylic acid esters suitable for use in the present invention include phthalates such as: Palatinol AH (Di-(2-ethylhexyl) phthalate; Palatinol AH L (Di-(2-ethylhexyl) phthalate); Palatinol C (Dibutyl phthalate); Palatinol IC (Diisobutyl phthalate); Palatinol N (Diisononyl phthalate); Palatinol Z (Diisodecyl phthalate); Palatinol 10-P (Di-(2-Propylheptyl) phthalate); Palatinol 711P (Heptylundecyl phthalate); Palatinol 911P (Nonylundecyl phthalate); Palatinol 11P-E (Diundecyl phthalate); Palatinol M (Dimethyl phthalate); Palatinol A (Diethyl phthalate); Palatinol A (Diethyl phthalate); and Palatinol K (Dibutylglycol phthalate). Further examples are the commercially available adipates such as: Plastomoll® DOA (Di-(2-ethylhexyl) adipate) and Plastomoll® DNA (Diisononyl adipate). Further examples of suitable commercially available materials are Vestinol C (DBP), Vestinol IB (DIBP), Vestinol AH (DEHP), Witamobm® 110 (610P) and Witamobm® 118 (810P) and Jayflex L9P and L11P.

In the process of the present invention, the hydrogenation is generally carried out at a temperature of from about 50 to 250° C., preferably from about 50 to 150° C., for example from about 80 to 130° C., in particular from about 105 to 120° C. The hydrogenation pressures used in the process of the present invention are generally above 10 bar, preferably from about 20 to about 300 bar, for example 30 to 200 bar, in particular 40 to 150 bar. Preferably the pressure is greater than 100 bars and more preferably greater than 130 bar. Generally, the hydrogenation is carried out with a stoichiometric hydrogen excess of 30 to 250%, preferably 50 to 200%, for example 100 to 150% in order to operate with a constant hydrogen off-gas rate.

The process of the present invention may be carried out either continuously or batch wise, with preference being given to carrying out the process continuously. Preferably, when the process is carried out continuously, the process is carried out in a fixed bed reactor, for example a down-flow reactor or a slurry reactor.

Batch reactors usually have a cylindrical shell with a spherical bottom head and a ring flange at the top. A closure head is bolted to this flange. Flange joints are well known to the skilled person and typically comprise two flanges with a gasket sandwiched between them. Leakage along the flange gasket due for example to thermal cycle fatigue by both temperature and pressure variation should be avoided. Different options, internal or external to the reactor, exist to mitigate this leaking flange problem.

According to one option a flexible box can be welded on the inside of the head flange to create a permanent seal. Various geometries may be used for the box. Internal box made with pipe is preferred to square box due to the elimination of the stress concentration at the corner joints that would make up the rectangular box. Also it avoids dead spaces within the reactor.

The box might be constructed as from the beginning of the construction of the reactor to prevent process fluid from leaking or later on to fix leakage problem without having to dismantle the flange joint. Preferably the internal box needs some flexibility to mitigate the effect of the temperature differentials that will occur during the operation.

Preferably extensions to the reactor wall are first welded at the desired location below and above the head flanges.

This method may be applied to joints with large deformations in the flanges. It is also applicable to any suitable flange joint, including but not limited to, full-face flange joint, narrow faced flange joint, flange joint formed from slip-on, screwed, socket weld, lap-joint and welding neck flanges and flanges joint formed from standard flanges.

It is also applicable to any reactor such as hydrogenation, polymerization, esterification, oxidation, and isomerization reactors.

Alternatively the process is carried out in a tubular reactor. Preferably, when the process is carried out continuously, the liquid volume flow rate in m³/hr divided by the known volume of catalyst in m³ (LVVH) is 1 to 5 hr⁻¹, preferably 2 to 5 hr⁻¹.

As hydrogenation gases, it is possible to use any gases which comprise free hydrogen and do not contain harmful amounts of catalyst poisons such as CO, CO₂, COS, H₂S and amines. For example, waste gases from a catalytic reformer can be used. Preference is given to using pure hydrogen as the hydrogenation gas.

The hydrogenation of the present invention can be carried out in the presence or absence of a solvent or diluent, i.e. it is not necessary to carry out the hydrogenation in solution. Preference is given to using a solvent or diluent. Any suitable solvent or diluent may be used. The choice is not critical as long as the solvent or diluent used is able to form a homogeneous solution with the benzenepolycarboxylic acid or derivate thereof to be hydrogenated. For example, the solvent or diluent may also comprise water, in particular the solvents or diluents may comprise water in an amount of 0.5 to 5 wt % based on the total weight of the feed stream. Preferably, the solvent or diluent is free of water.

Examples of suitable solvents or diluents include the following: straight-chain or cyclic ethers such as tetrahydrofuran or dioxane, and also aliphatic alcohols in which the alkyl radical preferably has from 1 to 10 carbon atoms, in particular from 3 to 6 carbon atoms. Examples of alcohols, which are preferably used, are i-propanol, n-butanol, i-butanol and n-hexanol. Preferably, the diluent comprises the hydrogenated product. Optionally, the diluent comprises light ends byproducts separated from the hydrogenated product. Preferably, the diluent comprises isoparaffinic fluids that can be easily separated from the hydrogenated product, such as isoparaffinic fluids available from ExxonMobil Chemical under the Isopar™ trade name Examples of suitable isoparaffinic fluids include Isopar™ C, Isopar E, Isopar G, and Isopar H, preferably Isopar C and Isopar E. Mixtures of these or other solvents or diluents can likewise be used.

The amount of solvent or diluent used is not restricted in any particular way and can be selected freely depending on requirements. However, preference is given to amounts which lead to a 10-70% strength by weight solution of the benzenepolycarboxylic acid or derivate thereof to be hydrogenated. For example, the amount of solvent or diluent used is from 30 to 300%, preferably 40-250%, more preferably 50 to 200% relative to the amount of benzenepolycarboxylic acid or derivative thereof used.

In the process of the present invention it is also possible to use one or more derivatives of benzenepolycarboxylic acids in the unpurified state that is in the presence of one or more starting materials for their manufacture such as for example alcohol in the case of ester derivatives. Also present may be traces of monoester derivatives, un-reacted acid such as phthalic acid, sodium monoester derivatives and sodium salts of the acids. In this aspect the benzenepolycarboxylic acid derivative is hydrogenated prior to purification and after hydrogenation is then sent to process finishing for stripping, drying and polishing filtration. In this aspect the benzenepolycarboxylic acid derivative may be an intermediate feed containing high levels of alcohol in the case of ester derivatives. There may be present 5 to 30% excess alcohol than that required to achieve complete esterification of the acid. In one embodiment there may be an intermediate feed containing 8 to 10 wt % isononyl alcohol in di-isononyl phthalate.

In the process of the present invention the desired products are one or more cyclohexyl materials derived from the hydrogenation of the corresponding benzenepolycarboxylic acid or derivatives thereof. Ideally the benzenepolycarboxylic acid or derivatives thereof are converted to the desired product with a high degree of selectivity and with the maximum conversion possible of the benzenepolycarboxylic acid or derivatives thereof.

Hydrogenations of this type often result in undesirable by-products of relatively low molecular weight and low boiling point; these by-products are referred to as “lights” or “light ends”. In the context of the present invention “lights” are defined as materials in the as hydrogenated reaction product that are eluted before the object cyclohexyl materials when the as hydrogenated reaction product is analyzed by Gas Liquid Chromatography. Details for one suitable method for determining the “lights” content of a product obtained by the process of the present invention is provided in EP 2 338 870 A1. When using the process of the present invention it is possible to obtain greater than 95% conversion of the starting material (one or more benzenepolycarboxylic acid or derivatives thereof), whilst at the same time producing less than 1.5% by weight based on the total weight of reaction product of “lights”. In the process of the present invention the product obtained directly from the hydrogenation reaction ideally contains the object cyclohexyl derivative(s) in an amount that equates to 97 or greater mole % conversion of the starting material, preferably 98.5 or greater mole % conversion, more preferably 99 or greater mole % conversion, and most preferably 99.9 or greater mole % conversion. In the process of the present invention the product obtained directly from the hydrogenation reaction ideally contains 1.3% or less, preferably 1.0% or less, more preferably 0.75% or less, even more preferably 0.5% or less, and in the most preferable embodiment less than 0.3% by weight based on the total weight of the reaction product of “lights”. When hydrogenated products of this level of purity are obtained it may be possible to use these materials directly in certain applications without the need for further purification of the as hydrogenated product such as plasticisers for plastics products.

The catalyst used in the present invention comprises one or more transition metals of Group VIII of the Periodic Table (previous IUPAC notation) deposited on one or more support materials. A particular preference is given to using rhodium, ruthenium, platinum, palladium, or mixtures thereof. A particularly preferred Group VIII metal is ruthenium. It has to be noted in this respect that besides one or more Group VIII metals other metals may be used in combination with the Group VIII metals such as Group IB, IIB or VIIB metals.

The metal content of the catalyst will vary according to its catalytic activity. Thus, the highly active noble metals may be used in smaller amounts than the less active base metals. For example, about 3 wt % or less or rhodium, ruthenium, palladium or platinum, based on the total weight of the catalyst, will be effective. The metal component may exceed about 30 wt % in a monolayer, based on the total weight of the monolayer.

Preferably, the catalyst comprises a Group VIII metal in an amount of from about 0.05 to 2.5 wt %, based on the total weight of the catalyst. For example, the catalyst comprises rhodium, ruthenium, platinum, palladium, or a mixture thereof in an amount of from 0.05 to 2.5 wt %, preferably 0.5 to 2.5 wt %, especially 0.9 to 2.1 wt %, based on the total weight of the catalyst. Optionally, the catalyst comprises ruthenium in an amount of from 0.05 to 2.5 wt %, preferably 0.5 to 2.5 wt %, especially 0.9 to 2.1 wt %, based on the total weight of the catalyst. Suitable methods for determining the metal content of the catalyst include, for example, mass balance during catalyst preparation, quantitative X-ray fluorescence analysis, atomic absorption or preferably inductively coupled plasma.

The catalyst used in the process of the invention comprises a support, for example a support comprising a porous inorganic material. Suitable support materials include silica, titanium dioxide, zirconium dioxide and alumina, for example theta-alumina. Preferably, the support material comprises silica or alumina. For example, the support material consists essentially of silica.

The support material with or without ruthenium and rhodium deposited thereon may be shaped into a wide variety of particle sizes. Optionally, the particles can be in the form of a powder, a granule, or a molded product, such as an extrudate. It may be that the support material is shaped into particles having an average diameter of from 0.5 to 5 mm Preferably, the support material is extruded to form particles having a length of 2-15 mm and a diameter of 1-2 mm A suitable method for determining the average diameter of particles is solid particle sieve analysis. Optionally, the shaped particles or extrudates have a size sufficient to pass through a 4 mesh (Tyler) screen and be retained on a 32 mesh (Tyler) screen. In cases where the catalyst is molded, such as by extrusion, the crystals can be extruded before drying or partially dried and then extruded.

The catalyst used in the present invention may be prepared by any method known in the art. For example, the catalyst may be prepared by impregnation of the support material with a solution of a salt of the Group VIII metal. Generally speaking, when the Group VIII metal is applied by impregnation of the support, the concentration of the solution and the duration of the impregnation process are chosen in order to achieve the desired catalyst metal content. The Group VIII metal may be applied to the support by steeping the support in aqueous Group VIII metal salt solution, by spraying appropriate metal salt solutions onto the support, or by other suitable methods. Suitable Group VIII metal salts for preparing the Group VIII metal salt solutions are the nitrates, nitrosyl nitrates, halides, carbonates, carboxylates, acetylacetonates, chloro complexes, nitro complexes or amine complexes of the corresponding Group VIII metals. Preference is given to ruthenium chloride. In the case of catalysts which have a plurality of active metals applied to the support, the metal salts or metal salt solutions can be applied simultaneously or in succession.

The catalyst used in the process of the invention comprises a halogen in an amount of from about 0.02 to 0.6% by weight, based on the weight of the catalyst. The halogen content can be determined by X-ray fluorescence analysis. Preferably, the halogen is chloride. Optionally, the catalyst may additionally comprise sodium in an amount of from 0.2 to 2% by weight, for example 0.5 to 1.6% by weight, based on the total weight of the catalyst. Sodium is measured by inductively coupled plasma.

The catalyst are preferably prepared by impregnating the support material once or more than once with a solution of a Group VIII metal halide, typically ruthenium chloride, alone or together with a solution of at least one further salts of metal of group IB, IIB or VIIB, drying the resulting solid and subsequent reduction. The solution of at least one further salts of metal are applicable in one or more impregnation steps together with the solution of Group VIII metal chloride or in one or more impregnation step separately from the solution of Group VIII metal chloride.

The concentration of the active metal precursor in the solutions depends, by its nature, upon the amount of active metal precursor to be applied and the adsorption capacity of the support material for the solution. It is usually less than 20% by weight, preferably from 0.01 to 6 wt % based on the total weight of the solution.

The impregnated support is then dried and subsequently reduced before being washed to achieve the desired content of halide.

The impregnated support is typically dried under standard pressure. The drying can also be promoted by employing reduced pressure. Frequently the drying is promoted by passing a gas stream over or through the material to be dried, for example in air or nitrogen.

The drying time is preferably in the range of from 1 to 30 h, preferably in the range of from 2 to 10 h.

The drying of the impregnated support is preferably carried out to such an extent that the content of water or volatile solvent before the subsequent reduction makes up less than 5 wt %, in particular not more than 2 wt % based on the total weight of the solid. The weight fractions specified relate to the weight loss of the solid determined at a temperature of 160° C., a pressure of 1 bar and a time of 10 min.

The solid obtained after the drying is converted to its catalytically active form by reducing the solid at temperatures generally in the range from 150° C. to 450° C., preferably 250° C. to 350° C., in a manner known per se.

For this purpose, the support impregnated support is contacted with hydrogen or a mixture of hydrogen and an inert gas at the above specified temperatures. Frequently the impregnated support is hydrogenated at standard hydrogen pressure in a hydrogen stream. Preference is given to effecting the reduction with movement of the solid, for example by reducing the solid in a rotary tube oven or a rotary sphere oven. The reduction can also be effected by means of organic reducing reagent such as hydrazine, formaldehyde, formates or acetates.

After reduction the catalyst is mild washed to achieve the required halogen content. Such step provide good hydrogenation performance while being economically advantageous

The washing step can be effected by contacting the reduced solid with a water while maintaining the temperature in a range from 40 to 80° C. For this purpose, catalyst is preferably contacted with deionized water at the above specified temperatures, the volume or weight ratio of catalyst with water being of 1/1 to 1/10, preferably from 1/2 to 1/5. Frequently the washing step is carried out at atmospheric pressure, optionally through a fixed bed. Preference is given to perform the washing with movement of the catalyst, for example in a rotary tube oven or a rotary sphere oven. The washing step may need to be repeated multiple times to achieve the required halogen content.

The drying step is the same as described above.

Preferably, in addition to the step of contacting a feed stream of benzenepolycarboxylic acid or derivative thereof with a hydrogen-containing gas in the presence of a catalyst under hydrogenation conditions to produce a hydrogenated product, the process comprises at least one of the following steps: i) transferring the hydrogenated product to one or more reactors; ii) separating excess hydrogen from the hydrogenated product; iii) subjecting the hydrogenated product to steam stripping, preferably to remove light ends from the hydrogenated product; iv) drying the hydrogenated product by nitrogen stripping under vacuum; and, v) subjecting the hydrogenated product to a filtration step. For example, the process may comprise at least two, at least three, at least four, or all five of steps i) to v).

Preferably, when the feed stream comprises a diluent or solvent, and when the diluent or solvent comprises water, the process comprises the step of drying the hydrogenated product by nitrogen stripping under vacuum.

Generally speaking, when the feed stream comprises a diluent or solvent, at least a portion of the diluent or solvent is recycled to the step of contacting a feedstream of benzenepolycarboxylic acid or derivative thereof with a hydrogen-containing gas in the presence of a catalyst under hydrogenation conditions to produce a hydrogenated product. Optionally, when the process comprises the step of steam stripping the hydrogenated product to remove light ends, the light ends are used as a diluent and are recycled to the step of contacting a feed stream of benzenepolycarboxylic acid or derivative thereof with a hydrogen-containing gas in the presence of a catalyst under hydrogenation conditions to produce a hydrogenated product.

Optionally, the hydrogenated product is subjected to gas/liquid separation, for example after cooling to a temperature of 20-50° C., to recover any excess hydrogen entrained in the product stream. It may be that separated excess hydrogen is recycled back to the hydrogenation reactor. Preferably, the hydrogenated product is filtered to remove any hydrogenation catalyst fines and then separated from byproducts formed during the hydrogenation process, for example using a continuous steam stripping column to remove light byproducts. Alternatively, it may be that a batch steam stripper is used. Optionally, the hydrogenated product is subjected to steam stripping at a temperature of 150-240° C. at reduced pressure, for example at a pressure of 50-900 mbara. Preferably, the steam to product ratio is in the range of in the range of 1-10%. It may be that the feed to the steam stripper is preheated using a feed/product heat exchanger, optionally followed by a steam preheating. Optionally, the steam stripped product is subjected to nitrogen stripping for removal of residual water. Preferably, the stripped, and optionally dried, product is filtered at a temperature of 70-120° C. Alternatively, the stripped, and optionally dried, product is subjected to treatment with an adsorbent as described in EP 1 663 940 and then filtered, optionally with the use of a filter aid. Any kind of filter can be used, such as cartridge, candle or plate filters, depending upon the quantity of solids to be removed.

Preferably, the hydrogenated product is subjected to a filtration step, wherein the hydrogenated product is filtered by contacting the hydrogenated product with a precoated filter or a cartridge filter.

The process of the present invention is further illustrated by means of the following non-limiting examples.

### EXAMPLES

### Example 1

Catalyst sample 1 containing 1% ruthenium on silica spheres was obtained from Johnson Matthey Catalysts, Orchard Road, Royston, Hertfordshire SG8 SHE, UK (refefrence 662B). The 3 mm silica spheres had a crush strength of 3.4 kg/mm, a dry bulk density of 0.504 kg/liter, a pore volume of 37 vol %, a surface area of 140 m²/g and an external void volume of 44 vol %. The catalyst contained 0.30 wt % chloride and 0.82 wt % sodium.

### Comparative Example 1

Comparative catalyst sample 1 was prepared by impregnation of Aerolyst 3041 silica with ruthenium nitrosyl nitrate using the incipient wetness method according to US2006/166809 and U.S. Pat. No. 7,595,420. The molar ratio of ruthenium to triethanolamine (TEA) was 20:1 and the resulting ruthenium content was 0.5 wt %. The particle size was 0.85-1.0 mm. The catalyst did not contain chloride or sodium.

### Example 2

Catalyst sample 2 containing 2% ruthenium on theta alumina trilobes with the product reference 662C was obtained from Johnson Matthey Catalysts. The 2.5 mm theta alumina trilobes had a crush strength of 1.35 kg/mm and a surface area of 110 m²/g. The catalyst contained 0.12 wt % chloride and 0.5 wt % sodium.

### Example 3

Continuous flow hydrogenation of Jayflex® DINP was performed with catalyst 1 at a pressure of 80 bar and at a temperature of 80° C. The weight of the catalyst was 2.6 g. The particles were crushed to a size of 0.85-1.0 mm Catalyst pretreatment was performed with a hydrogen flow rate of 30 ml/min at 80 bar and 200° C. for 19 hrs. Liquid feed flow rate was 10 g/hr, and the feed composition was 50% DINP and 50% Isopar C as diluent. The hydrogen flow rate was 20 ml/min. At startup the DINP conversion was about 95% and gradually declined over 8 days to 90% with 1100-1200 ppm light ends formation. In a parallel experiment the initial conversion at 80° C. was the same, but after 4 days the temperature was increased to 100° C. which gave steady conversions of 98-100% and 1300-1400 ppm light ends formation after 8 days on-stream.

### Comparative Example 3

Continuous flow hydrogenation of Jayflex DINP® was performed with comparative catalyst 1 at a pressure of 80 bar and at a temperature of 80° C. The weight of the catalyst was 3.0 g. Catalyst pretreatment was performed with a hydrogen flow rate of 30 ml/min at 80 bar and 200° C. for 19 hrs. Liquid feed flow rate was 10 g/hr, and the feed composition was 50% DINP and 50% Isopar C as diluent. The hydrogen flow rate was 20 ml/min. At startup the DINP conversion was 80% and gradually declined over 4 days to 70% with 950-1050 ppm light ends formation. After 4 days the temperature was increased to 100° C. which gave slowly declining conversions from 99 to 96% and 1300-1400 ppm light ends formation after 8 days on-stream. A further investigation demonstrated that the light ends formation correlated with the operating temperature rather than the DINP conversion.

### Example 4

Two-stage continuous flow hydrogenation of Jayflex DINP® was performed with catalyst 1 at a pressure of 150 bar and at a temperature of 115° C. The catalyst was activated by applying the maximum hydrogen flow rate at low pressure (1-5 bar), at 150° C. for 1 hour, and cool down to 100° C. while maintaining the hydrogen flow. The first stage was made up with four up-flow reactors in series with the first reactor containing pumice as a guard bed followed by three reactors with each 125 ml of catalyst 1. The first stage operated with a liquid throughput (VVH) of 2.0-4.5 ml/hr per ml of catalyst. The hydrogen off-gas flow was kept constant at 2 liter/min for all experiments (measured at ambient temperature and pressure), which is equivalent to a hydrogen stoichiometric excess of 100:1. The product from the first stage third reactor was separated in order to recycle part of the product back to the DINP feed. Typically the unit was operated with a 2 to 1 recycle flow. Hydrogen was added to the mixture of fresh and recycled DINP before entering the guard bed. At a VVH of 4.4 h⁻¹ including the 2 to 1 recycle the conversion was 80% with 99.75% selectivity to the hydrogenated DINP. At VVH of 2.0 h⁻¹ and 2/1 recycle the conversion was 95% with 99.55% selectivity. In a once-through operation at a VVH of 0.7 h⁻¹ the conversion became 98.5% with 99.65% selectivity. The first order hydrogenation activity for the lead reactor was on average 4.3 h⁻¹ and did not show any sign of deactivation during a run time of 900 ml fresh DINP per ml of catalyst. The rate increased through the reactor train from 3.7 h⁻¹ at around 70% conversion in tube 1 to 4.2 h⁻¹ at around 85% conversion in tube 2, and to 5.1 h⁻¹ at above 90% conversion in tube 3. At constant VVH of 4.4 h⁻¹ and 2/1 recycle the reactor temperature was varied and resulted in an increased activity which calculated into an activation energy of 38.9 kJ/mol.

### Example 5

The second stage had three up-flow reactors in series with each 125 ml of catalyst 1 and fed with the product at 95% conversion from the first stage hydrogenation. The catalyst was activated by applying the maximum hydrogen flow rate at low pressure (1-5 bar), at 150° C. for 1 hour, and cool down to 100° C. while maintaining the hydrogen flow. The second stage operated with a liquid throughput (VVH) of 0.9 ml/hr per ml of catalyst. Hydrogen was added to the second stage feed obtained from the first stage separator. The product from the second stage third reactor was separated and depressured for further product cleanup. The second stage hydrogenation section was operated with a hydrogen off-gas rate of 2 liter/min (measured at ambient temperature and pressure), which is equivalent to a hydrogen stoichiometric excess of 100:1. At 116° C. and 150 bar the rate constant was 4.4 h⁻¹ resulting in 360 wtppm residual DINP and 99.3 wt % purity. At 125° C. and 150 bar the rate constant was 7.9 h⁻¹ resulting in 2 wtppm residual DINP and 99.05 wt % purity. The light ends formation at 114° C. gradually decreased with increasing catalyst life. The effect of the temperature has been tested and resulted in an activation energy for light ends formation of 73.6 kJ/mol. The second stage reactor product composition showed the need for a stripper to remove the light ends:

| | | |
|---|---|---|
| C₈ and C₉ alkanes | 0.05 | all in wt % |
| Cyclohexanemethanol | 0.02 | |
| C₉ and C₁₀ Oxo alcohols | 0.60 | |
| Hexahydrophthalide | 0.15 | |
| 1,2-Cyclohexanedimethanol | 0.04 | |
| Monoesters and Oxo ethers | 0.10 | |
| Hydrogenated DINP | 99.05 | |
| Total | 100.01 | |

Most of the byproducts are easy to strip and recoverable, which results in a final product purity of 99.9 wt % hydrogenated DINP.

### Example 6

Continuous flow hydrogenation of Jayflex DINP was performed with catalyst 2 at a pressure of 150 bar and at a temperature of 105° C. The catalyst was tested in the same experimental set-up and conditions as described in Example 5. The hydrogenation activity of the catalyst 2 was twice of the activity of catalyst 1 in line with the double ruthenium metal content. The byproduct formation is for the theta alumina supported catalyst disadvantageously much higher than for the silica support material. At equal pressure of 150 bar and 115° C. reactor temperature the rate of light ends formation of theta alumina was 5-6 times higher than of silica. Reduction of pressure to 40 bar halved the byproduct formation. Reduction of operating temperature to 95° C. and pressure reduction to 40 bar resulted in an equal byproduct formation as for the silica supported catalyst at 150 bar and 115° C., but with a much lower conversion of DINP to the hydrogenated product.

### Comparative Example 4 (Check Number)

Comparative Catalyst sample 2 is a 1.52 wt % ruthenium on silica and was obtained from Johnson Matthey Catalysts, UK (reference 662D). The catalyst further contained 0.63 wt % chloride and 1.56 wt % sodium.

### Example 7

Catalyst 3 was obtained by water washing comparative catalyst sample 2 according to the procedure as described in [0056]. The catalyst contained 1.52 wt % ruthenium, 0.03 wt % chloride and 0.34 wt % sodium.

### Comparative Example 5 and Example 8

Continuous flow hydrogenation of Jayflex DINP was performed with comparative catalyst 2 and catalyst 3 at a pressure of 150 bar and at a temperature of 115° C. The catalysts were tested in the same experimental set-up and conditions as described in Example 5. At these conditions the rate constant for comparative catalyst 2 was 1.7 h⁻¹ while it was 5.0 h⁻¹ for catalyst 3. (note: no selectivity data are available for these experiments).

1.-15. (canceled)

16. A process for ring hydrogenation of a benzenepolycarboxylic acid or derivative thereof, which process comprises contacting a feed stream comprising said acid or derivative thereof with a hydrogen-containing gas in the presence of a catalyst under hydrogenation conditions to produce a hydrogenated product, wherein said catalyst comprises a Group VIII metal, a support material and a halogen, and wherein the halogen is present in an amount of from 0.02 to 0.60% by weight, based on the total weight of the catalyst.

17. A process according to claim 1, wherein the Group VIII metal is selected from a group consisting of rhodium, ruthenium, palladium and platinum, preferably ruthenium.

18. A process according to claim 1, wherein the Group VIII metal is present in an amount of from 0.05 to 2.5% by weight, based on the total weight of the catalyst.

19. A process according to claim 1, wherein the support material comprises a material selected from the group consisting of silica, titanium dioxide and alumina, preferably silica.

20. A process according to claim 1, wherein the halogen is present in an amount of from 0.03 to 0.50% by weight, preferably from 0.20 to 0.40% by weight, based on the total weight of the catalyst.

21. A process according to claim 1, wherein the halogen is chloride.

22. A process according to claim 1, wherein the process is carried out at a pressure of 20 to 220 bar, a temperature of 50 to 150° C., a LVVH of from 1 to 5 h⁻¹ and a hydrogen excess of 50 to 200%.

23. A process according to claim 1, wherein the process is carried out as a continuous process, preferably in a fixed bed reactor.

24. A process according to claim 1, wherein the feed stream additionally comprises a diluent, wherein the diluent is present in an amount of from 50 to 200 parts per 100 parts of the benzenepolycarboxylic acid or derivative thereof.

25. A process according to claim 1, wherein the feed stream additionally comprises water in an amount of from 0.5 to 5% by weight, based on the total weight of the feed stream.

26. A process according to claim 1, wherein the feed stream additionally comprises, and, optionally, one or more isoparaffinic fluids.

27. A process according to claim 1, wherein the process additionally comprises one or more of the following steps:
transferring the hydrogenated product to one or more reactors;
separating of excess hydrogen from the hydrogenated product;
subjecting the hydrogenated product to steam stripping;
drying the hydrogenated product by nitrogen stripping under vacuum; and,
subjecting the hydrogenated product to a filtration step.

28. A process according to claim 27, wherein the process comprises at least three of steps i) to v).

29. A process according to claim 27, wherein the process comprises all of steps i) to v).

30. A process according to claim 27, wherein the process comprises step v), and wherein the hydrogenated product is filtered by contacting the hydrogenated product with a precoated filter or a cartridge filter.
